(19) United States
(12) Patent Application Publication
(10) Pub. No.: US 2025/0368957 A1
(43) Pub. Date: Dec. 4, 2025

(54) METHOD FOR PRODUCING MATURE DENDRITIC CELLS

(71) Applicant: LIHPAO LIFE SCIENCE CORP., New Taipei City (TW)

(72) Inventors: Tzeon-Jye CHIOU, Taipei City (TW); Chun-Yu LIU, Taipei City (TW); Zi-Han HUANG, Kaohsiung City (TW); Yi-Pei CHENG, Taipei City (TW)

(21) Appl. No.: 18/876,664

(22) PCT Filed: Jun. 24, 2022
(86) PCT No.: PCT/CN2022/101069

§ 371 Date: Dec. 19, 2024

(51) Int. Cl. C12N 5/0784; C12N 5/00

(52) U.S. Cl. CPC C12N 5/0639; C12N 5/525; C12N 5/562; C12N 2501/22; C12N 2501/2304; C12N 2501/25

(57) ABSTRACT

Disclosed herein is a method for producing mature dendritic cells from peripheral blood mononuclear cells (PBMCs). The method includes the steps of, treating the PBMCs with a cultivating medium supplemented with interleukin 4 (IL-4) and granulocyte-macrophage colony-stimulating factor (GM-CSF) to produce immature dendritic cells; and then treating the immature dendritic cells with the cultivating medium supplemented with IL-4, GM-CSF, tumor necrosis factor alpha (TNF-α), and Prostaglandin E₂ (PGE₂) to produce the mature dendritic cells. According to embodiments of the present disclosure, the PBMCs used in the present method are isolated from a leukocyte concentrate or a cryopreserved peripheral blood stem cells (PBSCs) stock.

### BACKGROUND OF THE INVENTION

### 1. Field of the Invention

The present disclosure relates to a method for producing mature dendritic cells. More particularly, the present disclosure relates to a method for producing mature dendritic cells from peripheral blood mononuclear cells (PBMCs).

### 2. Description of Related Art

Dendritic cells (DCs) are found in various tissues, where they detect homeostatic imbalances and present antigens to T cells, so as to establish a link between innate and adaptive immune responses. DCs are present in two different functional states, “mature” and “immature,” which are distinguished by many features, however, the ability to activate antigen-specific naïve T cells in secondary lymphoid organs is the hallmark of mature DCs. Hence, mature DCs play a key role in the activation of the immune system by acting as potent antigen-presenting cells, and this pivotal position, along with their abilities to generate dendritic cells from monocytes and uptake of antigen, allow DCs to serve as a vehicle for immunotherapy of a variety of indications.

Various methods for generating clinical mature DCs have been developed these years; among them, deriving DCs from monocytes is the most common approach. To this purpose, monocytes are collected from human donors and are cultivated in the presence of cytokines (e.g., interleukin (IL)-4) and growth factors (e.g., granulocyte-macrophage colony-stimulating factor (GM-CSF)). However, there are some problems in clinical need to be addressed.

Typically, monocytes collected from human peripheral blood must be enriched prior to culturing, in which lymphocytes, red blood cells, and platelets are depleted. Monocyte enrichment from peripheral blood mononuclear cells (PBMCs) is often achieved by plastic adherence; however, this process is an open multi-flask process with a risk of contamination, thus, it is not an ideal process for large-scale production in which current Good Manufacturing Practices (cGMP) is required.

Alternatively, monocyte enrichment may be achieved by counterflow centrifugal elutriation (CCE) cell separation, which relies on centrifugal force and the counterflow drag force to collect cells in fractions as they pass through the centrifuge, thus enables separation of cells based on size differences. Similar to plastic adherence approach stated above, the elutriation process also runs the risk of cross-contamination, rendering the process not suitable for large-scale production.

In view of the foregoing, there exists in the related art a need of an improved method for producing mature dendritic cells in an efficient and mass-produced manner.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a method for producing mature dendritic cells from peripheral blood mononuclear cells (PBMCs). The method comprises steps of (a) treating the PBMCs with a cultivating medium supplemented with interleukin 4 (IL-4) and granulocyte-macrophage colony-stimulating factor (GM-CSF) to produce immature dendritic cells; and (b) treating the immature dendritic cells of step (a) with the cultivating medium supplemented with IL-4, GM-CSF, tumor necrosis factor alpha (TNF-α), and Prostaglandin E₂ (PGE₂) to produce the mature dendritic cells. In the present method, the PBMCs are isolated from a leukocyte concentrate or a cryopreserved peripheral blood stem cells (PBSCs) stock.

In some preferred embodiments, the leukocyte concentrate may be freshly collected from a subject.

According to some embodiments of the present disclosure, the cryopreserved PBSCs stock is produced by (i) mixing freshly isolated PBSCs and an antifreeze to produce a PBSCs stock; and (ii) subjecting the produced PBSCs stock of step (i) to a freezing treatment in a chamber, in which the ambient temperature of the chamber is decreased from about 4° C. to about −95° C. within about 55 to 70 minutes.

Preferably, in step (ii) stated above, the ambient temperature of the chamber is decreased stepwise by, (i) from about 4° C. to −7° C. within 21 to 25 minutes, (ii) from about −7° C. to −25° C. within 5 to 6 minutes, (iii) from −25° C. to −45° C. within 25 to 30 minutes, and (iv) from about −45° C. to −95° C. within 4 to 9 minutes.

According to some embodiments of the present disclosure, the cryopreserved PBSCs stock may be stored in a liquid nitrogen.

In alternative or additional embodiments, prior to step (a), the present cryopreserved PBSCs stock is subjected to a thawing treatment at a first temperature about 37° C., followed by at a second temperature about 0 to 5° C. accompanied by a low speed of centrifuge until the cryopreserved PBSCs are thawed.

According to some embodiments of the present disclosure, the cultivating medium may comprise L-glutamine, streptomycin sulfate, and gentamicin sulfate. According to alternative embodiments, the cultivating medium may comprise salts, sugars, amino acids, vitamins, transferrin, albumin, and insulin.

According to some embodiments, in step (a) of the present method, the IL-4 and the GM-CSF are present in a ratio about 1:1 to 2:1 by unit in the cultivating medium.

In some preferred embodiments, the cultivating medium used in step (a) is further supplemented with a serum. In some working examples, the serum is an autologous serum.

According to some embodiments, in step (b) of the present method, the IL-4, the GM-CSF, and the TNF-α are present in a ratio of 1:1:1 by unit in the cultivating medium.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawing, where:

FIG. 1 is a flow diagram depicting the present method 10 according to one embodiment of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

The term “cultivating medium” as used herein refers to culture media that are commonly used in cell cultivation. The components of media may vary depending on the type of cells to be cultured, which is well known in the art. For example, a standardized cancer cell line medium includes proteinogenic or non-proteinogenic amino acids, saccharides, salts, and/or trace elements capable of supporting and ensuring continuous cancer cell proliferation in vitro; a standardized cell medium consisting of amino acids, antibiotics, cytokines, sugars, and proteins is required in mononuclear cell cultivation. Detail ingredients of specific cultivating medium can be obtained from public information provided by suppliers or can be found in public documents. According to some embodiments of the present disclosure, the cultivating medium used in the present disclosure comprises L-glutamine, streptomycin sulfate, and gentamicin sulfate. According to other embodiments of the present disclosure, the cultivating medium mainly includes salts, sugars, amino acids, vitamins, and human proteins (e.g., transferrin, albumin, and insulin).

The term “subject” as used herein refers to a mammal that can serve as cell donors for the present method. The term “mammal” refers to all members of the class Mammalia, including humans, primates, domestic and farm animals, such as rabbit, pig, sheep, and cattle; as well as zoo, sports or pet animals; and rodents, such as mouse and rat; preferably humans. Further, the term “subject” intended to refer to both the male and female gender unless one gender is specifically indicated.

### 2. Detail Description of Preferred Embodiments

The present disclosure is based, at least in part, on the discovery of enriched mature dendritic cells may be produced and derived from certain cell populations by a series of treatments, so as to increase the productivity and quality of the mature dendritic cells. Accordingly, the present disclosure aims at providing a novel method for producing mature dendritic cells from peripheral blood mononuclear cells (PBMCs) that have been isolated from a leukocyte concentrate or a cryopreserved peripheral blood stem cells (PBSCs) stock.

Accordingly, the objective of the present disclosure is directed to a method of producing mature dendritic cells from PBMCs. The method comprises at least following steps: (a) treating the PBMCs with a cultivating medium supplemented with interleukin 4 (IL-4) and granulocyte-macrophage colony-stimulating factor (GM-CSF) to produce immature dendritic cells; and (b) treating the immature dendritic cells of step (a) with the cultivating medium supplemented with IL-4, GM-CSF, tumor necrosis factor alpha (TNF-α), and prostaglandin E₂ (PGE₂) to produce the mature dendritic cells. For this objective, the PBMCs used in the present method are isolated from a leukocyte concentrate or a cryopreserved PBSCs stock.

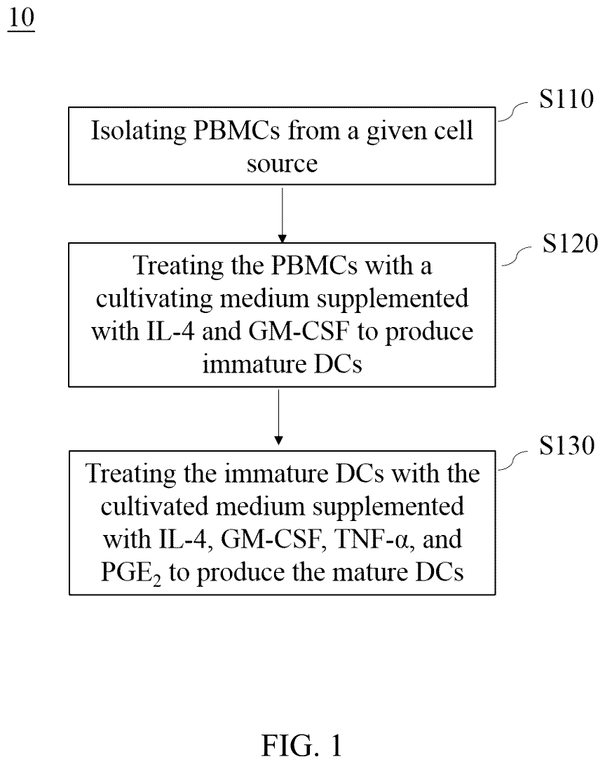

Reference is made to FIG. 1, which is a flow diagram illustrating the steps of the present method 10 for producing mature dendritic cells. Specifically, the method 10 at least includes three steps, which are: isolating PBMCs from a given cell source (step S110); treating the PBMCs with a cultivating medium supplemented with IL-4 and GM-CSF to produce immature dendritic cells (step S120); and treating the immature dendritic cells with the cultivating medium supplemented with IL-4, GM-CSF, TNF-α, and PGE₂ to produce mature dendritic cells (step S130). According to the present disclosure, the PBMCs used in the present method are isolated from a leukocyte concentrate that is freshly collected from a subject, or are isolated from a cryopreserved PBSCs stock that has been stored in a designated environment for a time. The isolation and production procedures from two PBMCs sources will be described in detail below.

2.1 PBMCs Isolated from Leukocyte Concentrates

According to some embodiments of the present disclosure, the leukocyte concentrate may be freshly harvested from subjects via methods well known in the art. For example, blood may be freshly drawn from a subject, and is immediately subjected to cell separation via gradient centrifugation in a cell separator to produce the desired PBMCs (S110). Examples of the subject that may serve as the donor of leukocyte concentrate include, but are not limited to, a human, a mouse, a rat, a hamster, a guinea pig, a rabbit, a dog, a cat, a cow, a goat, a sheep, a monkey, a horse, and etc. Preferably, the subject is a human. In a general procedure for producing leukocyte concentrates, human whole blood is collected in a blood bag that contains anticoagulants (e.g., acid citrate dextrose (ACD)), the collected whole blood is then centrifuged to produce three layers of materials, which are the plasma, a buffy-coat, and erythrocytes. After removing the plasma layer, the buffy-coat layer is collected and is termed as the leukocyte concentrates. According to embodiments of the present disclosure, the leukocyte concentrates may be used immediately after isolation, or may be stored for at least 4 hours, such as 4, 8, 12, 16, 20, and 24 hours until usage. In one working example, the leukocyte concentrate is stored for no longer than 24 hours before PBMCs are isolated therefrom.

2.2 PBMCs Isolated from Cryopreserved PBSCs Stocks

According to alternative embodiments of the present disclosure, the PBMCs used for producing mature dendritic cells are isolated from a PBSCs stock that has been stored at a sub-zero temperature for certain period of time. In some embodiments, the cryopreserved PBSCs stock has been stored in liquid nitrogen for one, two, three, four, five, six, seven, eight, nine, or ten months, or over one, two, three, four, five, six, seven, eight, nine, or ten years. In one preferred embodiment, the cryopreserved PBSCs stock has been stored in liquid nitrogen for over three years before being used for the isolation of PBMCs. In some working examples, the cryopreserved PBSCs stock has been stored for three, four, five, six or eight years.

In some embodiments, the cryopreserved PBSCs stock is substantially produced by steps of, (i) mixing freshly isolated PBSCs and an antifreeze to produce a PBSCs stock; and (ii) subjecting the produced PBSCs stock of step (i) to a freezing treatment in a chamber, in which the ambient temperature of the chamber is decreased from about 4° C. to about −95° C. within about 55 to 70 minutes. In some working examples, the PBSCs are first collected and isolated from human donors by means and/or tools well known in the art, which include but are not limited to, semiautomated or automated cell separators, and cell centrifuges. The collected PBSCs are then mixed with the antifreeze that helps prevent cells from freezing at freezing-temperature, thereby producing a PBSCs stock that may tolerate subsequent freezing treatment, which is normally conducted in a sub-zero chamber (e.g., a cryobiology freezer).

Examples of antifreeze suitable for use in this procedure induce, but are not limited to, antifreeze agents (e.g., ethylene glycol, propylene glycol, propylene glycol methyl ether, dimethyl sulfoxide (DMSO), and 2-ethylhexanoic acid (2-EHA)); antifreeze glycoproteins (e.g., insect antifreeze proteins (AFPs) such as TmAFP or CfAFP; fish AFPs Type I to Type IV; plant AFPs; sea ice organism AFPs such as EfcIBP); and a combination thereof. In one working example, the antifreeze is DMSO.

Once the PBSCs stock is placed into the sub-zero chamber, the ambient temperature of the chamber is decreased stepwise from about 4° C. to about −95° C. within a specified time period under a manual or an automatic control. Specifically, the freezing procedure can be automatically conducted by a computer program pre-implemented in the cryobiology freezer, and the ambient temperature in the chamber varies stepwise (i) from about 4° C. to −7° C. within 21 to 25 minutes, such as 21, 22, 23, 24, or 25 minutes; (ii) from about −7° C. to −25° C. within 5 to 6 minutes, such as, 5.1, 5.2, 5.3, 5.4, 5.5, 5.6, 5.7, 5.8, 5.9, or 6 minutes; (iii) from −25° C. to −45° C. within 25 to 30 minutes, such as 25, 26, 27, 28, 29, or 30 minutes; and finally (iv) from about −45° C. to −95° C. within 4 to 9 minutes, such as 4, 5, 6, 6.1, 6.2, 6.3, 6.4, 6.5, 6.6, 6.7, 6.8, 6.9, 7, 8, or 9 minutes. In one working example, the ambient temperature in the chamber is set to decrease stepwise, (i) from about 4° C. to −7° C. in 22 minutes, (ii) from about −7° C. to −25° C. in 5.68 minutes; (iii) from −25° C. to −45° C. within 28.57 minutes; and finally (iv) from about −45° C. to −95° C. within 6.5 minutes.

Additionally or optionally, to increase the efficiency of isolating the PBMCs, the cryopreserved PBSCs stock may be subjected to a thawing treatment before starting the isolation. In some embodiments, the thawing treatment includes thawing the cryopreserved PBSCs stock first at about 37° C., then at about 0 to 5° C., independently accompanied by a low speed of centrifuge until the cryopreserved PBSCs are completely thawed. The thawing treatment may be conducted in a manner well known in the art. For example, the cryopreserved PBSCs stock is thawed by placing the stock first in a heated bath that has a constant temperature of 37° C., and then in a refrigerated centrifuge where the temperature is set to be 4° C. Meanwhile, a low speed of centrifuge is applied to the cryopreserved PBSCs stock to precipitate the PBMCs that have been thawed.

2.3 Production of Dendritic Cells from PBMCs

Reference is still made to FIG. 1, after the PBMCs are collected from either sources indicated above, they are then cultivated in a cultivating medium supplemented with a cytokine and a growth factor under a given condition well-known and widely-used in the art, so as to produce immature dendritic cells (step S120). In some embodiments, the PBMCs are cultivated in a cultivating medium supplemented with interleukin 4 (IL-4) and granulocyte-macrophage colony-stimulating factor (GM-CSF) at 37° C. in a 5-10% CO₂ incubator for about one to eight days, such as, one, two, three, four, five, six, seven, or eight days. In some working examples, the PBMCs are initially suspended in a basal medium on day 0, then on day 3 and 6, the basal medium is replaced by another fresh basal medium having additionally added IL-4 and GM-CSF, and the entire cultivation lasts for a total of 7 days. In other working examples, the PBMCs are initially suspended in a basal medium on day 0, then on day 3, the basal medium is replaced by another fresh basal medium having additionally added IL-4 and GM-CSF, and the entire cultivation lasts for a total of 5 days.

The thus-produced immature dendritic cells are then cultivated in another cultivating medium, which is supplemented with IL-4, GM-CSF, tumor necrosis factor alpha (TNF-α), and Prostaglandin E₂ (PGE₂), thereby producing mature dendritic cells (step S130). Similar to the step S120, the immature dendritic cells thus harvested may be resuspended in the same cultivating medium and cultivated in a chamber atmosphere of 5-10% CO₂ at 37° C. for a few days until mature dendritic cells are produced. In some working examples, the mature dendritic cells are found after the immature DCs have been cultivated for 2 days.

The cultivating media respectively used in steps S120 and S130 of the present method 10 may have same or different components and/or ingredients capable of supporting cell growth and proliferation. In some embodiments, the cultivating media used in the afore-two steps (S120 and S130) have same components; in alternative embodiments, the components of the cultivating medium in step S120 differ from those in step S130. Examples of components and/or ingredients that can be comprised in the present cultivating medium include, but are not limited to, proteinogenic or non-proteinogenic amino acids, saccharides, salts, trace elements, antibiotics, cytokines, vitamins, human proteins, and a combination thereof. In one working example, the present cultivating medium is mainly composed of L-glutamine, streptomycin sulfate and gentamicin sulfate. In another working example, the cultivating medium is mainly composed of salts, sugars, amino acids, vitamins, transferrin, albumin, and insulin.

According to some embodiments, when used in the present method, cytokines and/or growth factors including IL-4, GM-CSF, TNF-α, and PGE₂, are respectively added to the cultivating medium in pre-designated amounts. In some embodiments, in step S120, the cultivating medium is supplemented with IL-4 and GM-CSF, in which the IL-4 and GM-CSF are present in a unit ratio about 1:1 to 2:1; for example, about 1:1, 1.1:1, 1.2:1, 1.3:1, 1.4:1, 1.5:1, 1.6:1, 1.7:1, 1.8:1, 1.9:1 or 2:1. In one working example, the IL-4 and GM-CSF are present in a unit ratio about 1:1; in another working example, the IL-4 and GM-CSF are present in a unit ratio about 1.6:1. On the other hand, in step S130 of the present method 10, the cultivating medium is supplemented with IL-4, GM-CSF, TNF-α, and/or PGE₂; in one working example, the cultivated medium is supplemented with IL-4, GM-CSF, and TNF-α, in which the IL-4, GM-CSF, and TNF-α are present in a ratio of 1:1:1 by unit in the cultivated medium of step S130. Alternatively or optionally, a serum may be added into the cultivating medium as well. In one specific working example, the cultivating medium used in step S120 is further supplemented with an autologous serum at a concentration of 1%.

After being cultivated in sequence in the cultivating medium described in steps S120 and S130, mature dendritic cells can be successfully produced from PBMCs.

By the virtue of the above features, the present method can produce mature dendritic cell in an efficient manner, which in turn increases the cell productivity and quality.

### EXAMPLES

### Materials and Methods

### Cell Sources

### Leukocyte Concentrates

Leukocyte concentrates were freshly harvested from three healthy human donors with the aid of cell separator (SPECTRA OPTIA, Terumo BCT) under informed consent, and then stored at 15-25° C. until further use.

### Peripheral Blood Stem Cells (PBSCs) Stocks

Whole blood was collected from healthy human donors under informed consent and was stored in blood bags for subsequent separation of plasma and a buffy-coat layer containing peripheral blood stem cells (PBSCs) by centrifuging the whole blood at 10° C., 1500 rpm for 10 minutes. The plasma was transferred to new, empty blood bag and mixed with dimethyl sulfoxide (DMSO) and stored at 4° C. for at least 20 minutes, then the mixture was re-mixed with the buffy coat to form a PBSC stock, which was temporally stored at 4° C. no more than six hours.

PBSCs stock was then brought to cryopreservation in a cryobiology freezer (IceCube 14M-A automatic freezer, Minitube) with a pre-implemented program within a stretch of 65 minutes, in which the ambient temperature in the chamber was decreased stepwise according to a pre-designated scheme described in Table 1.

TABLE 1
Designated temperatures and time for cryopreservation of PBSCs

| Temp (° C.) | Time (min) | Slope (° C./min) |
|---|---|---|
| 4.00 | 0.00 | — |
| −7.00 | 22.00 | −0.5 |
| −55.00 | 1.93 | −24.83 |
| −55.00 | 0.75 | 0.00 |
| −25.00 | 3.00 | 10.00 |
| −45.00 | 28.57 | −0.7 |
| −60.00 | 3.00 | −5.00 |
| −95.00 | 3.50 | −10.00 |
| −95.00 | 20.00 | 0.00 |

After cryopreservation, the cryopreserved PBSCs were stored in cryogenic chambers filled with liquid nitrogen for three, four, five, six, or eight years, respectively.

### Serum Preparation

Plasma isolated above was centrifuged at 2,115 rpm for 30 minutes (4° C.). Supernatant was collected and heated in a water bath at 56° C. for 30 minutes, let cool down at room temperature for 10 minutes, and then centrifuged again at the same speed and temperature for another 30 minutes. The thus produced supernatant contained serum.

Immature Dendritic Cells (iDCs) Produced from Leukocyte Concentrates

Freshly collected leukocyte concentrates were dispensed into centrifuge tubes (maximum volume: 50 ml), each tube contained about 10 ml leukocyte concentrates, which was diluted by adding another 10 ml of Dulbecco's phosphate-buffered saline (DPBS). The thus formed leukocyte solution was transferred slowly to another centrifuge tube coated with Ficoll-Paque (Cytiva), and centrifuged at 1,800 rpm, for 30 minutes at 24° C. After centrifugation, the buffy-coat containing peripheral blood mononuclear cells (PBMCs) was collected and transferred to new tubes, washed with DPBS at 37° C. and resuspended in basal media (Thermo Fisher-Gibco), and the cell numbers were counted.

The collected PBMCs were first cultivated in basal media for 1 to 2 hours, then washed with DPBS at 37° C., cells were then divided into two fractions (Fractions I and II) and respectively subjected to further cultivation according to conditions as follows: Fraction I: AIM-V medium (Gibco) supplemented with 1000 IU/ml of interleukin 4 (IL-4) and 1000 IU/ml of granulocyte-macrophage colony-stimulating factor (GM-CSF), in a CO₂ incubator at 37° C. for seven days; Fraction II: DC medium (CellGenix) supplemented with 800 IU/ml of IL-4, 500 IU/ml of GM-CSF, and 1% of serum, in a CO₂ incubator at 37° C. for at least five days. Cells were harvested on days 5 or 7 for cell phenotypes examination.

Immature DCs Produced from Cryopreserved PBSCs Stocks

Cryopreserved PBSCs stocks were placed in a water bath at 37° C. until thawed. The thawed PBSCs were harvested and transferred into centrifuge tubes containing DPBS (9×PBSCs volume), and centrifuged at 1,300 rpm at 4° C. for 10 minutes. PBSCs pellet was then resuspended in DPBS for total nucleated cell count (TNC).

Based on the cell count (or TNC number), the thus collected PBSCs were dispensed into centrifuge tubes at the concentration of 5×10⁷ to 2×10⁸ cells/20 mL DPBS per tube. The thus formed solution was transferred to a new centrifuge tube coated with Ficoll-Paque, and then centrifuged for 30 minutes (1800 rpm/24° C.). After centrifugation, the buffy-coat containing PBMCs was collected and transferred into new tubes for further treatment.

The collected PBMCs were first cultivated in basal media for 1 to 2 hours, washed with DPBS at 37° C., and then continued to cultivate in AIM-V medium supplemented with 1000 IU/ml of IL-4, and 1000 IU/ml of GM-CSF in a CO₂ incubator at 37° C. for seven days. Cells were harvested on day 7 for cell phenotypes investigation.

Mature DCs Produced from iDCs

IDCs produced from leukocyte concentrates or cryopreserved PBSCs stocks as stated above were harvested and collected at a density of 5×10⁶ cells per fraction. Supernatant of each fraction was removed via centrifugation, and the remaining iDCs pellet was resuspended in AIM-V medium. Then, the resuspended iDCs were cultivated in the AIM-V medium supplemented with 1000 IU/ml of IL-4, 1000 IU/ml of GM-CSF, 1000 IU/ml of tumor necrosis factor alpha (TNF-α), and 1 μM of Prostaglandin E₂ (PGE₂) at 37° C. for two days. Cells were harvested on day 3 for cell phenotypes and potency tests.

### PBSCs Viability

The thawed PBSCs were collected and sorted with anti-CD34 and anti-CD45 antibodies to separate stem cells (i.e., PBSCs) from non-stem cells (i.e., differentiated leukocytes), and the viability of present PBSCs was determined by the equation of, (Number of viable stem cells/number of viable leukocytes)×100%.

### Phenotypic Assessment

Cells were harvested, resuspended in DPBS, incubated for 10 minutes with human FcR blocking reagent, and then incubated for 10 minutes with anti-human CD14-FITC, CD34-PE, CD40-PE, CD45-FITC, CD80-PE, CD83-FITC, CD86-PE, and/or HLA-DR-FITC (BD Biosciences) antibodies in the dark. The stained cells were washed and blocked three to five times by cell staining buffer (5% FBS in DPBS), and 5 μL of 7-Aminoactinomycin D (7-AAD) were added to react with cells for 10 minutes. Afterwards, cells were sorted by flow cytometry (FACSCanto II, BD Biosciences).

### Endocytic Activity

IDCs and/or DCs (2×10⁵ cells per centrifuge tube having round bottom) were incubated in water bath at 37° C. for 20 minutes with 1 μL of FITC labeled dextran (Sigma), blocked at 4° C., then incubated in the dark for further 20 minutes. The stained cells were washed and blocked five to seven times by cell staining buffer (5% FBS in DPBS). Anti-CD11 antibody (5 L) and 7-Aminoactinomycin D (7-AAD, 5 L) were sequentially added to cells, which were sorted by flow cytometry.

### Flow Cytometry Analysis

Cell sorting was achieved by flow cytometry using fluorochrome-conjugated antibodies. After a final wash prior to sorting, cells were filtered through a 40 m nylon cell strainer (Becton Dickinson, USA) to remove cell clumps, and then sorted with flow cytometry (FACSCanto II, BD Bioscience). Sorted populations were respectively collected in complete media (5% FBS in DPBS) until further use.

### Bacterial Endotoxin Detection

Endotoxin was detected by using endotoxin detection system (BioTek) in accordance with the manufacturer's instruction and guidance provided in U.S. pharmacopeia.

### Biosafety Assessment

Whole cellular DNA was extracted and Mycoplasma was detected using Mycoplasma detection kit (Sartorius) in accordance with the manufacturer's instruction.

### Method Suitability and Sterility Test

The present cell products were collected and filtered, and then subjected to cultivation of standard bacteria and fungi species (i.e., Aspergillus brasiliensis ATCC 16404, Bacillus subtilis subsp. spizizenii ATCC 6633, Candida albicans ATCC 10231, Clostridium sporogenes ATCC 19404, Pseudomonas aeruginosa ATCC 9027, and Staphylococcus aureus subsp. aureus ATCC 6538) in accordance with guidance provided in U.S. pharmacopeia.

### IDCs Productivity

PBMCs on day 0 and immature dendritic cells (iDCs) on days 5 or 7 were respectively collected and counted, and the productivity of immature dendritic cells produced by the present method was determined by the following equation:

$$[ cell numbers ( days 5 or 7 ) \times iDC \% ] / [ cell number ( day 0 ) \times PBMC \% ] \times 100 \% = Yield of iDCs ( \% ) .$$

### Example 1 Stability of the Cryopreserved Peripheral Blood Stem Cells (PBSCs) Stocks

In this example, whether the present PBSCs could retain their abilities in differentiating into peripheral blood mononuclear cells (PBMCs) after long-term cryopreservation was investigated.

To this purpose, PBSCs were first cryopreserved for at least three years and then thawed at 37° C. in according with procedures described in “Materials and Methods” section, thereby gave rise to thawed PBSCs. Four replicates of thawed PBSCs were independently co-cultivated with anti-CD34-PE and anti-CD45-FITC antibodies, and the phenotypes thereof were verified by flow cytometry.

The cultivated cells derived from cryopreserved PBSCs stocks were abundant with CD34⁺ cells (i.e., the thawed PBSCs after being cryopreserved for longer than three years, particularly for three, four, five, six, or eight years) with an average viability larger than 90%, indicating that the present cryopreservation procedure could preserve PBSCs for at least 3 years without adversely changing their pluripotent nature.

### Example 2 Yields of Immature Dendritic Cells (iDCs) Produced from PBSCs Collected and Cultivated by the Present Method

In this example, the yield of iDCs derived from three groups (Groups I to III) of PBMCs were investigated, in which each Group of PBMCs were collected and cultivated at conditions as described in “Materials and Methods” section. Specifically, PBMCs of Group I were derived from leukocyte concentrates and cultivated in cultivating medium supplemented with IL-4 and GM-CSF; PBMCs of Group II were derived from leukocyte concentrates and cultivated in cultivating medium supplemented with IL-4, GM-CSF, and serum (1%); and PBMCs of Group III were derived from cryopreserved PBSCs stocks and cultivated in cultivating medium supplemented with IL-4 and GM-CSF. The productivity or yield of iDCs from each PMBCs group were determined.

It was found that, Groups II and III independently had a significantly higher productivity in iDCs. Specifically, the yields of iDCs from Group I (i.e., PBMCs cultivated in a serum-free medium) and Group II (i.e., PBMCs cultivated in a serum-addition medium) were about 4.43% and 10%, respectively. Further, the yield of iDCs from Group II was more than two-folds of that of Group I, indicating that the addition of serum significantly improved the yields of iDCs. On the other hand, the yields of iDCs from Group III PBMCs (i.e., PBMCs derived from PBSCs stocks) was about 20.5%, which was significantly higher than those of iDCs derived from Groups I and II, indicating that the present cryopreservation procedure could preserve pluripotent nature and differentiability of PBSCs. The data collectively indicated that the present method can achieve a high production of iDCs.

### Example 3 Characterization of DCs Produced by the Present Method

### 3.1 Phenotype

In this experiment, whether the cell products of the present method were indeed immature and/or mature dendritic cells was investigated via examination of their phenotypes. To this purpose, the present immature and mature DCs products (i.e., the present immature and mature DCs produced according to procedures described “Materials and Methods” section) were mixed with anti-human CD14, CD40, CD80, CD83, CD86, and HLA-DR antibodies in dark, and cells recognized by each afore-indicated antibody were sorted by flow cytometry. Quantitative results of phenotypic profiles are provided in Table 2.

TABLE 2
Quantitative results of phenotypic profiles

| | | | Present | Acceptance |
|---|---|---|---|---|
| | Present iDCs | Acceptance | mDCs | criteria |
| Biomarkers | products | criteria of iDCs | products | of mDCs |
| CD14 | 3.1% | <60% | 3.6% | <60% |
| CD40 | 60.7% | — | 82.5% | Level up |
| CD86 | 74.9% | >60% | 99.6% | >60% |
| CD80 | 46.0% | — | 87.3% | Level up |
| CD83 | 4.8% | — | 84.2% | Level up |
| HLA-DR | 93.1% | >60% | 99.5% | >60% |

The data in Table 2 evidenced that the present cell products were substantially immature or mature dendritic cells.

### 3.2 the Endocytic Ability

In this experiment, the endocytic ability of the DCs produced by the present method was investigated. To this purpose, the present immature and mature DCs produced according to procedures described in “Materials and Methods” section were respectively co-cultivated with fluorescent dextrans for 20 minutes. The endocytic ability of cells was verified by measuring the proportion of endocytosed to non-endocytosed dextrans in iDCs and/or DCs with the aid of flow cytometry.

It was found that, the endocytic activity of iDCs was 61.5% (greater than 20%), while that of DCs was lower than 20%, such finding complied with the requirements in acceptance criteria (AC) of industrial dendritic cell products. The data indicated that the present method may successfully produce desire dendritic cell products.

### 3.3 Safety

In this study, whether the produced DCs complied with the safety requirements of clinical needs was investigated. To this purpose, the levels of endotoxin and mycoplasma in the DCs were determined via procedures described in “Materials and Methods” section.

It was found that, the level of endotoxin in the present DCs products was lower than 0.25 EU/ml, which is much lower than cGMP regulations (<2 EU/ml); and the DCs was negative for the presence of mycoplasma. These results collectively confirmed the present DCs products are safe for clinics and human bodies.

### 3.4 Method Suitability and Sterility

In this study, whether the produced DCs complied with the sterile requirements of cGMP regulations was investigated. To this purpose, produced DCs were collected and filtered, then subjected to microbial cultivation according to procedures described in “Materials and Methods” section.

It was found that the present DC products were negative for any presence of microorganisms, which confirmed the present DCs products comply with the cGMP regulations.

Taken together, the data depicted in Examples 1 to 3 collectively indicated that the present method can shorten the DCs' producing time and increase their productivity, therefore has an outstanding merit.

It will be understood, that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for producing mature dendritic cells from peripheral blood mononuclear cells (PBMCs), comprising:
(a) treating the PBMCs with a cultivating medium supplemented with interleukin 4 (IL-4) and granulocyte-macrophage colony-stimulating factor (GM-CSF) to produce immature dendritic cells; and
(b) treating the immature dendritic cells of step (a) with the cultivating medium supplemented with IL-4, GM-CSF, tumor necrosis factor alpha (TNF-α), and prostaglandin E₂ (PGE₂) to produce the mature dendritic cells,
wherein, the PBMCs are isolated from a leukocyte concentrate or a cryopreserved peripheral blood stem cells (PBSCs) stock.

2. The method of claim 1, wherein the leukocyte concentrate is freshly collected from a subject.

3. The method of claim 1, wherein the cryopreserved PBSCs stock is produced by (i) mixing freshly isolated PBSCs and an antifreeze to produce a PBSCs stock; and (ii) subjecting the produced PBSCs stock of step (i) to a freezing treatment in a chamber, in which the ambient temperature of the chamber is decreased from about 4° C. to about −95° C. within about 55 to 70 minutes.

4. The method of claim 3, wherein the ambient temperature of the chamber is decreased stepwise (i) from about 4° C. to −7° C. within 21 to 25 minutes, (ii) from about −7° C. to −25° C. within 5 to 6 minutes, (iii) from −25° C. to −45° C. within 25 to 30 minutes, and (iv) from about −45° C. to −95° C. within 4 to 9 minutes.

5. The method of claim 1, wherein the cryopreserved PBSCs stock is stored in a liquid nitrogen.

6. The method of claim 1, wherein prior to step (a), the cryopreserved PBSCs stock is thawed at a first temperature about 37° C., then at a second temperature about 0 to 5° C., independently accompanied by a low speed of centrifuge until the cryopreserved PBSCs are completely thawed.

7. The method of claim 1, wherein the cultivating medium comprises L-glutamine, streptomycin sulfate, and gentamicin sulfate.

8. The method of claim 1, wherein the cultivating medium comprises salts, saccharides, amino acids, vitamins, transferrin, albumin, and insulin.

9. The method of claim 1, wherein in step (a), the IL-4 and the GM-CSF are present in a ratio about 1:1 to 2:1 by unit in the cultivating medium.

10. The method of claim 9, wherein in step (a), the cultivating medium is further supplemented with a serum.

11. The method of claim 10, wherein the serum is an autologous serum.

12. The method of claim 1, wherein in step (b), the IL-4, the GM-CSF, and the TNF-α are present in a ratio of 1:1:1 by unit in the cultivating medium.
